(19) United States
(12) Patent Application Publication
(10) Pub. No.: US 2026/0014069 A1
(43) Pub. Date: Jan. 15, 2026

(54) UV double-sided adhesive that can be photocuring twice

(71) Applicants: Zhang Moran, Yiwu City (CN); Shenzhen Baohe New Materials R&D Co., Ltd., Shenzhen (CN)

(72) Inventors: Duan Wenwen, Shenzhen (CN); Zhang Moran, Yiwu City (CN)

(21) Appl. No.: 19/031,220

(22) Filed: Jan. 17, 2025

(30) Foreign Application Priority Data
Jul. 9, 2024 (CN) 202410914031.1

(51) Int. Cl. A61K 8/81; A61K 8/85; A61K 8/87; A61Q 3/02

(52) U.S. Cl. CPC A61K 8/8152; A61K 8/85; A61K 8/87; A61Q 3/02; A61K 2800/594; A61K 2800/654; A61K 2800/81

(57) ABSTRACT

A UV double-sided adhesive that can be photocuring twice is provided, which is provided with a first PET release film, a glue layer, and a second PET release film in sequence. Adding fluorine modified core-shell polyacrylic acid modified resin to the glue layer can improve the photopolymerization stability of the glue layer of the present disclosure, and shorten the photopolymerization time during photocuring in practical use; on the other hand, while ensuring excellent adhesion in practical use, it is waterproof, easy to peel off, and leaves no marks. The UV double-sided adhesive has excellent adhesion, faster photocuring speed, and stable photocuring effect, it is waterproof, easy to peel off during cleaning, and does not leave marks. It solves problems of not being able to quickly attach and firmly wear, improves a tedious process of cleaning residual adhesive, enhances the wearing experience, and reduces labor costs in nail salons.

### CROSS-REFERENCE TO RELATED APPLICATIONS

This application claims priority to Chinese Patent Application No. 202410914031.1, filed on Jul. 9, 2024, which is hereby incorporated by reference in its entirety.

### TECHNICAL FIELD

The present disclosure relates to the field of nail glue technologies, and in particular, to a UV double-sided adhesive that can be photocuring twice.

### BACKGROUND

At present, materials used for nail wearing and nail art on the market are generally ordinary removable double-sided adhesive nano tape. This type of double-sided adhesive is in the form of small pieces, each piece is the same size as the nail, and is called jelly glue on the market. The bonding way is to clean the nail and then apply the double-sided adhesive to the nail. The advantage of this type of double-sided adhesive is that it can be quickly applied, taking about 3-5 minutes each time. This double-sided adhesive will peel off under dynamic external forces and has poor waterproof performance, not reaching a completely firm state. There is another bonding way, which is to use canned or tubular solid UV glue. When using, take one piece and place it on the nail, then flatten it. There will be residual glue overflowing from the roots and fingertips, and each finger will be individually illuminated with UV light. Cleaning these residual glue and lighting will take 30-50 minutes. The advantage of this adhesive is that it adheres firmly and is waterproof, but the nail sticking time is slow, taking 30-50 minutes. At present, jelly glue is quick to stick to nails, but not strong enough. UV solid adhesive is firm, but it sticks slowly.

The fluorine atoms in organic fluorine compounds determine their unique properties. Fluorine is the element with the highest electronegativity in the periodic table. Its small radius, long C—F bond length, high bond energy, and spiral distribution of fluorine atoms connected to the main chain of fluorinated polymers along serrated C—C bonds provide a tight shield for the polymer main chain from direct external factors, thereby improving the weather resistance, oxidation resistance, and corrosion resistance of organic fluoropolymers. Organic fluorine compounds have low intermolecular cohesion, small molecular forces between air and polymer interfaces, low surface free energy, and low surface friction coefficient, endowing organic fluorine polymers with excellent water resistance, oil resistance, and wear resistance.

### SUMMARY

The purpose of the present disclosure is to provide a UV double-sided adhesive that can be photocuring twice to solve problems raised in the background technology. The aim is to provide a nail adhesive with excellent adhesion, faster photocuring speed, more stable photocuring effect, waterproof, easy to peel off during cleaning, and no marks left.

To achieve the above objectives, the present disclosure provides the following technical solution:

A UV double-sided adhesive that can be photocuring twice, including a first PET release film, a glue layer, and a second PET release film that are arranged in sequence;
the glue layer is obtained by UV curing adhesive through photocuring;
the UV curing adhesive is consisted of component A and component B;
the component A is a pre-polymerized resin blend compound including the following components by weight:
5-6 parts of fluorine modified core-shell polyacrylic acid modified resin, 25-30 parts of polyurethane acrylic resin, 25-30 parts of tackifying acrylic resin, 8.3-10 parts of isobornyl acrylate, and 16.7-20 parts of ethylhexyl acrylate;
the component B is an auxiliary component including the following components by weight:
1-3 parts of surfactant, 0.83-1 parts of antioxidant, 1.67-2 parts of coupling agent, 2.5-3 parts of photoinitiator.

In some embodiments of the present disclosure, the first PET release film and the second PET release film are biaxially oriented polyester films with a thickness of 25-50 μm; the glue layer has a thickness of 0.5±0.01 mm.

In some embodiments of the present disclosure, the tackifying acrylic resin is polymethyl methacrylate; the photoinitiator is one of 1-hydroxycyclohexylphenylketone, 2,4-dihydroxybenzophenone, α-diethoxy acetophenone, and benzophenone.

In some embodiments of the present disclosure, the surfactant is disproportionated rosin potassium soap or maleic rosin.

In some embodiments of the present disclosure, preparation steps of the fluorine modified core-shell polyacrylic acid modified resin are as follows:
S1: adding a hybrid emulsifier and deionized water into a round bottom flask, stirring at room temperature, dropping 20 g of core material into the flask for 5 min, stirring vigorously for 1 h, and obtaining a pre-polymerized core material lotion;
S2: adding SR-10 emulsifier, AEO-9 emulsifier, NaHCO₃, ammonium persulfate into a mixture composed of the pre-polymerized core material lotion and deionized water; stirring at 80° C. and 220 r/min to synthesis a seed lotion, and keeping at 85° C. for 30 min; dropping the pre-polymerized modified shell material lotion to the seed lotion until to be blue, and staying at 85° C. for 30 min; dropping the pre-polymerized modified shell material lotion and an aqueous solution of initiator with the same experimental procedure, cooling to room temperature to obtain the fluorine modified core-shell polyacrylic acid modified resin.

It should be noted that due to the high hydrophobicity, extremely low surface energy, and self-aggregation characteristics of fluorinated groups, when dodecafluoroheptyl methacrylate is used as a shell monomer in the polymerization reaction, they aggregate on the surface of latex particles, rendering it easier for fluorinated fragments to aggregate on the surface of the film. This enables the present disclosure to be easily peeled off from nails with hydrophobic keratin as the main component, without leaving any marks, while ensuring excellent adhesion in practical use. At the same time, due to the high bonding energy of C—F bonds in long-chain fluoroalkyl chains, they can shield thermal energy and protect the core polymer chain. Therefore, an introduction of dodecafluoroheptyl methacrylate improves the photopolymerization stability of the glue layer of the present disclosure, resulting in shorter and more stable photopolymerization time during secondary curing in practical use. The introduction of a small amount of dodecafluoroheptyl methacrylate as a shell material has improved the hydrophobicity and photopolymerization rate of the UV double-sided adhesive. The application of core-shell structure not only enhances the performance of UV double-sided adhesive, but also reduces a consumption of fluorinated monomers, rendering the synthesized UV double-sided adhesive cost-effective and environmentally friendly.

In some embodiments of the present disclosure, the core material is one of methyl methacrylate, butyl acrylate, or acrylic acid; the modified shell material is dodecafluoroheptyl methacrylate.

In some embodiments of the present disclosure, in S1, a mass ratio of SR-10 emulsifier to AEO-9 emulsifier in the hybrid emulsifier is 2:1, a mass ratio of hybrid emulsifier, deionized water, and core material is 2:3:1; in S2, a mass ratio of SR-10 emulsifier, AEO-9 emulsifier, NaHCO₃, ammonium persulfate, pre-polymerized modified shell material lotion and deionized water is 0.25:0.13:0.2:0.15:6.5:44.

In some embodiments of the present disclosure, preparation steps of the UV curing adhesive are as follows:
S1: adding 5-6 parts by weight of fluorine modified core-shell polyacrylic acid modified resin, 25-30 parts by weight of polyurethane acrylic resin, 25-30 parts by weight of tackifying acrylic resin, 8.3-10 parts by weight of isobornyl acrylate, and 16.7-20 parts by weight of ethylhexyl acrylate to a reactor to obtain a pre-polymerized resin blend compound;
S2: adding 1-3 parts by weight of surfactant dropwise to the pre-polymerized resin blend compound at 0-5° C., homogenizing for 30 minutes, heating up to 50-60° C. and refluxing for 6 hours, adding 0.83-1 parts by weight of antioxidant and 1.67-2 parts by weight of coupling agent, reacting for 3-4 hours at 90-120° C., cooling to 50-60° C., and obtaining a crude adhesive;
S3: adding 2.5-3 parts by weight of photoinitiator and water to the crude adhesive for compounding, homogenizing for 30 minutes, and preparing the UV curing adhesive.

In some embodiments of the present disclosure, a production environment configuration: a production workshop needs to be equipped with a yellow light to shield natural light and UV light sources.

In some embodiments of the present disclosure, a preparation method of the UV double-sided adhesive includes the following steps:
S1: pouring evenly mixed glue into a UV curing coating machine, turning on UV curing lamp, adjusting light energy to 500-600 mJ, setting a feeding speed to 3.1 m/min, evenly applying the glue onto the first PET release film by a machine, forming the glue layer after the glue is solidified for a first time, and covering the second PET release film on the other side of the glue layer;
S2: material winding, rolling into a film with a width of 1000 mm and a length of 200 m by a winding machine;
S3: cutting the film into small rolls and feeding them onto a die-cutting machine for die-cutting into small pieces; the particle size of each piece is consistent with sizes of nails on human hands, thereby achieving a perfect fit.

Compared with the existing technology, the present disclosure has the following beneficial effects.

On the one hand, due to the high bonding energy of C—F bonds in long-chain fluoroalkyl chains, they can shield thermal energy and protect the core polymer chain. Therefore, the introduction of dodecafluoroheptyl methacrylate improves the photopolymerization stability of the glue layer of the present disclosure, resulting in shorter and more stable photopolymerization time during secondary curing in practical use. On the other hand, due to the high hydrophobicity, extremely low surface energy, and self-aggregation characteristics of fluorinated groups, the present disclosure ensures excellent adhesion in practical use while being waterproof, easy to peel off, and leaving no marks. The introduction of a small amount of dodecafluoroheptyl methacrylate as a shell material has improved the hydrophobicity and photopolymerization rate of UV double-sided adhesive. The application of core-shell structure not only enhances the performance of UV double-sided adhesive, but also reduces the consumption of fluorinated monomers, rendering the synthesized UV double-sided adhesive cost-effective and environmentally friendly.

The present disclosure provides a nail polish adhesive with excellent adhesion, faster photocuring speed, more stable photocuring effect, waterproof, easy to peel off and leave no marks during cleaning, which solves a problem of not being able to quickly adhere and firmly put on the nail, improves a tedious process of cleaning residual adhesive, enhances the wearing experience, and reduces labor costs in nail salons.

### DESCRIPTION OF EMBODIMENTS

Below, the technical solutions in the embodiments of the present disclosure will be clearly and completely described in combination with the accompanying drawings. Obviously, the described embodiments are only a part of the embodiments of the present disclosure, not all of them. Based on the embodiments of the present disclosure, all other embodiments obtained by those skilled in the art without creative work are within the protection scope of the present disclosure.

Preparation steps of fluorine modified core-shell polyacrylic acid modified resin are as follows:
S1: adding a hybrid emulsifier and deionized water into a round bottom flask, stirring at room temperature, dropping 20 g of methyl methacrylate into the flask for 5 min, stirring vigorously for 1 h, and obtaining a pre-polymerized core material lotion; preparing a pre-polymerized modified shell material lotion with dodecafluoroheptyl methacrylate as a modified shell material under the same experimental procedure; a mass ratio of SR-10 emulsifier to AEO-9 emulsifier in the hybrid emulsifier is 2:1, and a mass ratio of the hybrid emulsifier, the deionized water, and core material is 2:3:1;
S2: adding 0.25 g of SR-10 emulsifier, 0.13 g of AEO-9 emulsifier, 0.2 g of NaHCO₃, 0.15 g of ammonium persulfate into a mixture of 6.5 g of the pre-polymerized core material lotion and 44 g of deionized water; stirring at 80° C. and 220 r/min to synthesis a seed lotion, and keeping at 85° C. for 30 min; dropping the pre-polymerized modified shell material lotion to the seed lotion until to be blue, and staying at 85° C. for 30 min; dropping the pre-polymerized modified shell material lotion and an aqueous solution of initiator with the same experimental procedure, cooling to room temperature to obtain the fluorine modified core-shell polyacrylic acid modified resin.

A UV double-sided adhesive that can be photocuring twice includes a first PET release film, a glue layer, and a second PET release film that are arranged in sequence.

Production environment configuration: a production workshop needs to be equipped with a yellow light to shield natural light and UV light sources.

A preparation method of the UV double-sided adhesive that can be photocuring twice includes the following steps:
S1: pouring evenly mixed glue into a UV curing coating machine, turning on UV curing lamp, adjusting light energy to 500-600 mJ, setting a feeding speed to 3.1 m/min, evenly applying the glue onto the first PET release film by a machine, forming a glue layer after the glue is solidified for a first time, and covering the second PET release film on the other side of the glue layer;
S2: material winding, rolling into a film with a width of 1000 mm and a length of 200 m by a winding machine;
S3: cutting the film into small rolls and feeding them onto a die-cutting machine for die-cutting into small pieces; particle size of each piece is consistent with the size of the nails on the human hand, thereby achieving a perfect fit.

### EXAMPLE 1

the glue layer is obtained by UV curing adhesive through photocuring, and the UV curing adhesive is consisted of component A and component B;
component A is a pre-polymerized resin blend compound including the following components by weight: 6 parts of fluorine modified core-shell polyacrylic acid modified resin, 30 parts polyurethane acrylic resin, 30 parts of polymethyl methacrylate, 10 parts of isobornyl acrylate, and 20 parts of ethylhexyl acrylate;
component B is an auxiliary component including the following components by weight: 3 parts of disproportionated rosin potassium soap, 1 part of antioxidant, 2 parts of silane coupling agent KH-560, and 3 parts of 1-hydroxycyclohexylphenylketone.

Preparation steps of the UV curing adhesive are as follows:
S1: adding 6 parts by weight of fluorine modified core-shell polyacrylic acid modified resin, 30 parts by weight of polyurethane acrylic resin, 30 parts by weight of polymethyl methacrylate, 10 parts by weight of isobornyl acrylate, and 20 parts by weight of ethylhexyl acrylate into a reactor to obtain a pre-polymerized resin blend compound;
S2: adding 3 parts by weight of disproportionated rosin potassium soap dropwise to the pre-polymerized resin blend compound at 5° C., homogenizing for 30 minutes, heating up to 50-60° C. and refluxing for 6 hours, adding 1 part by weight of antioxidant 1010 and 2 parts by weight of silane coupling agent KH-560, reacting at 90-120° C. for 3-4 hours, cooling to 50-60° C., and obtaining a crude adhesive;
S3: adding 3 parts by weight of 1-hydroxycyclohexylphenylketone and 20 parts by weight of water to the crude adhesive for compounding, homogenizing for 30 minutes, and preparing the UV curing adhesive.

### EXAMPLE 2

the glue layer is obtained by UV curing adhesive through photocuring, and the UV curing adhesive is consisted of component A and component B;
component A is a pre-polymerized resin blend composed including the following components by weight: 5.5 parts of fluorine modified core-shell polyacrylic acid modified resin, 27.5 parts of polyurethane acrylic resin, 27.5 parts of polymethyl methacrylate, 9.2 parts of isobornyl acrylate, and 18.4 parts of ethylhexyl acrylate;
component B is an auxiliary component including the following components by weight: 2 parts of disproportionated rosin potassium soap, 0.92 parts of antioxidant 1010, 1.84 parts of silane coupling agent KH-560, 2.75 parts of 1-hydroxycyclohexylphenylketone.

Preparation steps of the UV curing adhesive are as follows:
S1: adding 5.5 parts by weight of fluorine modified core-shell polyacrylic acid modified resin, 27.5 parts by weight of polyurethane acrylic resin, 27.5 parts by weight of polymethyl methacrylate, 9.2 parts by weight of isobornyl acrylate, and 18.4 parts by weight of ethylhexyl acrylate to a reactor to obtain a pre-polymerized resin blend compound;
S2: adding 2 parts by weight of disproportionated rosin potassium soap dropwise to the pre-polymerized resin blend compound at 5° C., homogenizing for 30 minutes, heating up to 50-60° C. and refluxing for 6 hours, adding 0.92 parts by weight of antioxidant 1010 and 1.84 parts by weight of silane coupling agent KH-560, reacting at 90-120° C. for 3-4 hours, cooling to 50-60° C., and obtaining a crude adhesive;
S3: adding 2.75 parts by weight of 1-hydroxycyclohexylphenylketone and 20 parts by weight of water to the crude adhesive for compounding, homogenizing for 30 minutes, and preparing the UV curing adhesive.

### EXAMPLE 3

the glue layer is obtained by UV curing adhesive through photocuring, and the UV curing adhesive is consisted of component A and component B;
component A is a pre-polymerized resin blend compound including the following components by weight: 5 parts of fluorine modified core-shell polyacrylic acid modified resin, 25 parts of polyurethane acrylic resin, 25 parts of polymethyl methacrylate, 8.3 parts of isobornyl acrylate, and 16.7 parts of ethylhexyl acrylate;
component B is an auxiliary component including the following components by weight: 1 part of disproportionated rosin potassium soap, 0.83 parts of antioxidant 1010, 1.67 parts of silane coupling agent KH-560, 2.5 parts of 1-hydroxycyclohexylphenylketone.

Preparation steps of the UV curing adhesive are as follows:
S1: adding 5 parts by weight of fluorine modified core-shell polyacrylic acid modified resin, 25 parts by weight of polyurethane acrylic resin, 25 parts by weight of tackifying acrylic resin, 8.3 parts by weight of isobornyl acrylate, and 16.7 parts by weight of ethylhexyl acrylate to a reactor to obtain a pre-polymerized resin blend compound;
S2: adding 1 part by weight of disproportionated rosin potassium soap dropwise to the pre-polymerized resin blend compound at 5° C., homogenizing for 30 minutes, heating up to 50-60° C. and refluxing for 6 hours, adding 0.83 parts by weight of antioxidant 1010 and 1.67 parts by weight of silane coupling agent KH-560, reacting at 90-120° C. for 3-4 hours, cooling to 50-60° C., and obtaining a crude adhesive;
S3: adding 2.5 parts by weight of 1-hydroxycyclohexylphenylketone and 20 parts by weight of water to the crude adhesive for compounding, homogenizing for 30 minutes, and preparing the UV curing adhesive.

### COMPARISON EXAMPLE 1

the glue layer is obtained by UV curing adhesive through photocuring, and the UV curing adhesive is consisted of component A and component B;
component A is a pre-polymerized resin blend compound including the following components by weight: 6 parts of perfluoro heptyl methacrylate, 30 parts of polyurethane acrylic resin, 30 parts of polymethyl methacrylate, 10 parts of isobornyl acrylate, 20 parts of ethylhexyl acrylate;
component B is an auxiliary component including the following components by weight: 3 parts of disproportionated rosin potassium soap, 1 part of antioxidant 1010, 2 parts of silane coupling agent KH-560, and 3 parts of 1-hydroxycyclohexylphenylketone.

Preparation steps of the UV curing adhesive are as follows:
S1: adding 6 parts by weight of fluorine modified core-shell polyacrylic acid modified resin, 30 parts by weight of polyurethane acrylic resin, 30 parts by weight of polymethyl methacrylate, 10 parts by weight of isobornyl acrylate, and 20 parts by weight of ethylhexyl acrylate to a reactor to obtain a pre-polymerized resin blend compound;
S2: adding 3 parts by weight of disproportionated rosin potassium soap dropwise to the pre-polymerized resin blend compound at 5° C., homogenizing for 30 minutes, heating up to 50-60° C. and refluxing for 6 hours, adding 1 part by weight of antioxidant 1010 and 2 parts by weight of silane coupling agent KH-560, reacting at 90-120° C. for 3-4 hours, cooling to 50-60° C., and obtaining a crude adhesive;
S3: adding 3 parts by weight of 1-hydroxycyclohexylphenylketone and 20 parts by weight of water to the crude adhesive for compounding, homogenizing for 30 minutes, and preparing the UV curing adhesive.

A difference between this comparative Example and Example 1 is that fluorine modified core-shell polyacrylic acid modified resin was not used, but dodecafluoroheptyl methacrylate as the shell material was used to prepare the fluorine modified core-shell polyacrylic acid modified resin.

### COMPARISON EXAMPLE 2

the glue layer is obtained by UV curing adhesive through photocuring, and the UV curing adhesive is consisted of component A and component B;
component A is a pre-polymerized resin blend compound including the following components by weight: 6 parts of methyl methacrylate, 30 parts of polyurethane acrylic resin, 30 parts of polymethyl methacrylate, 10 parts of isobornyl acrylate, 20 parts of ethylhexyl acrylate;
component B is an auxiliary component including the following components by weight: 3 parts of disproportionated rosin potassium soap, 1 part of antioxidant 1010, 2 parts of silane coupling agent KH-560, 3 parts of 1-hydroxycyclohexylphenylketone.

Preparation steps of the UV curing adhesive are as follows:
S1: adding 6 parts by weight of fluorine modified core-shell polyacrylic acid modified resin, 30 parts by weight of polyurethane acrylic resin, 30 parts by weight of polymethyl methacrylate, 10 parts by weight of isobornyl acrylate, and 20 parts by weight of ethylhexyl acrylate into a reactor to obtain a pre-polymerized resin blend compound;
S2: adding 3 parts by weight of disproportionated rosin potassium soap dropwise to the pre-polymerized resin blend compound at 5° C., homogenizing for 30 minutes, heating up to 50-60° C. and refluxing for 6 hours, adding 1 part by weight of antioxidant 1010 and 2 parts by weight of silane coupling agent KH-560, reacting at 90-120° C. for 3-4 hours, cooling to 50-60° C., and obtaining a crude adhesive;
S3: adding 3 parts by weight of 1-hydroxycyclohexylphenylketone and 20 parts by weight of water to the crude adhesive for compounding, homogenizing for 30 minutes, and preparing the UV curing adhesive.

A difference between this Comparative Example and Example 1 is that fluorine modified core-shell polyacrylic acid modified resin was not used, but instead methyl methacrylate was used as the core material for preparing the fluorine modified core-shell polyacrylic acid modified resin.

### COMPARISON EXAMPLE 3

the glue layer is obtained by UV curing adhesive through photocuring, and the UV curing adhesive is consisted of component A and component B;
component A is a pre-polymerized resin blend composed including the following components by weight: 30 parts of polyurethane acrylic resin, 30 parts of polymethyl methacrylate, 10 parts of isobornyl acrylate, 20 parts of ethylhexyl acrylate;
component B is an auxiliary component including the following components by weight: 3 parts of disproportionated rosin potassium soap, 1 part of antioxidant 1010, 2 parts of silane coupling agent KH-560, 3 parts of 1-hydroxycyclohexylphenylketone.

Preparation steps of the UV curing adhesive are as follows:
S1: adding 30 parts by weight of polyurethane acrylic resin, 30 parts by weight of polymethyl methacrylate, 10 parts by weight of isobornyl acrylate, and 20 parts by weight of ethylhexyl acrylate to a reactor to obtain a pre-polymerized resin blend compound;
S2: adding 3 parts by weight of disproportionated rosin potassium soap dropwise to the pre-polymerized resin blend compound at 5° C., homogenizing for 30 minutes, heating up to 50-60° C. and refluxing for 6 hours, adding 1 part by weight of antioxidant 1010 and 2 parts by weight of silane coupling agent KH-560, reacting at 90-120° C. for 3-4 hours, cooling to 50-60° C., and obtaining a crude adhesive;
S3: adding 3 parts by weight of 1-hydroxycyclohexylphenylketone and 20 parts by weight of water to the crude adhesive for compounding, homogenizing for 30 minutes, and preparing the UV curing adhesive.

A difference between this Comparative Example and Example 1 is that no fluorine modified core-shell polyacrylic acid modified resin was added.

### Testing

### 1. Adhesive Performance Testing

A UV curing lamp was used to irradiate the UV double-sided adhesive, and after complete photocuring, a BLD-100S electronic peel strength tester was used to test the cyclic viscosity and peel strength according to standard PSTC-6 and PSTC-1, and the residual adhesive situation was observed.

### 2. Photocuring Rate Test

A UV curing lamp was used to irradiate the UV double-sided adhesive, light energy was adjusted to 500-600 mJ, and its complete photocuring rate was tested. The photocuring rate is characterized by solid conversion rate (W), and the calculation formula is as follows:

$$G 1 = ( M 3 - M 1 ) / ( M 2 - M 1 ) \times 100 \%$$

G1—solid content;
M1—weight of aluminum foil tray;
M2—weight of UV double-sided adhesive before drying;
M3—weight of UV double-sided adhesive before and after drying;

$$W = G 1 / G 2 \times 100 \%$$

W—solid conversion rate;
G2—theoretical solid content.

### 3. Waterproof Performance Test

At room temperature, contact angle (WCA) of water was measured using a handleless drop method with a contact angle goniometer. The area used is 4 cm², and the test is conducted at 6-7 different positions on a surface of the UV double-sided adhesive. The average values are taken.

### 5. Summary of Test Results

TABLE 1

| | | | | Solid | |
|---|---|---|---|---|---|
| | Cyclic viscosity | 180° Peel strength | Is there any | conversion rate | Contact |
| | (N/25 mm) | (N/25 mm) | residual glue | (%) | angle (° C.) |
| Example 1 | 6.23 ± 0.15 | 2.81 ± 0.17 | No | 100 | 145.8 |
| Example 2 | 6.25 ± 0.14 | 2.74 ± 0.19 | No | 100 | 145.4 |
| Example 3 | 6.24 ± 0.17 | 2.89 ± 0.18 | No | 100 | 145.6 |
| Comparative | 5.86 ± 0.20 | 3.25 ± 0.13 | Yes (some | 94.5 | 130.7 |
| Example 1 | | | residual glue) | | |
| Comparative | 5.45 ± 0.18 | 3.64 ± 0.15 | Yes (more | 91.4 | 114.5 |
| Example 2 | | | residual glue) | | |
| Comparative | 5.03 ± 0.23 | 4.16 ± 0.12 | Yes (more | 87.6 | 103.9 |
| Example 3 | | | residual glue) | | |

### 6. Summary

1. The results of Examples 1-3 show that the solid conversion rate of the UV double-sided adhesive that can be photocuring twice prepared by the present disclosure is 100%. This indicates that the photocuring rate is fast, ensuring excellent adhesion performance while maintaining moderate peel strength. There is no residual adhesive left during peeling, and the contact angle is above 145°, demonstrating excellent waterproof performance.
2. Compared with Comparative Examples 1-3, Examples 1-3 showed significant improvements in all data except for the 180° peel strength, indicating that the addition of fluorine modified core-shell polyacrylic acid modified resin enhances the adhesion performance of the UV double-sided adhesive, accelerates the photocuring speed, and improves the waterproof effect.
3. Due to the presence of fluorinated organic compounds with low surface energy characteristics in the formula, the performance of Comparative Example 1 is superior to Comparative Examples 2 and 3 in all aspects. The introduction of a small amount of dodecafluoroheptyl methacrylate as the shell material improves the hydrophobicity and photopolymerization rate of the UV double-sided adhesive, but the performance of Comparative Example 1 is still slightly inferior to Examples 1-3. The application of core-shell structure not only improves the performance of UV double-sided adhesive, but also reduces the consumption of fluorinated monomers, rendering the synthesized UV double-sided adhesive cost-effective and environmentally friendly.

Although the embodiments of the present disclosure have been shown and described, it will be understood by those skilled in the art that various changes, modifications, substitutions, and variations can be made to these embodiments without departing from the principles and spirit of the present disclosure. The scope of the present disclosure is limited by the appended claims and their equivalents.

What is claimed is:

1. A UV double-sided adhesive that can be photocuring twice, comprising a first PET release film, a glue layer, and a second PET release film that are arranged in sequence;
the glue layer is obtained by UV curing adhesive through photocuring;
the UV curing adhesive is consisted of component A and component B;
the component A is a pre-polymerized resin blend compound comprising the following components by weight:
5-6 parts of fluorine modified core-shell polyacrylic acid modified resin, 25-30 parts polyurethane acrylic resin, 25-30 parts of tackifying acrylic resin, 8.3-10 parts of isobornyl acrylate, and 16.7-20 parts of ethylhexyl acrylate;
the component B is an auxiliary component comprising the following components by weight:
1-3 parts of surfactant, 0.83-1 parts of antioxidant, 1.67-2 parts of coupling agent, and 2.5-3 parts of photoinitiator.

2. The UV double-sided adhesive according to claim 1, wherein the first PET release film and the second PET release film are biaxially oriented polyester films with a thickness of 25-50 μm; the glue layer has a thickness of 0.5±0.01 mm.

3. The UV double-sided adhesive according to claim 1, wherein the tackifying acrylic resin is polymethyl methacrylate;
the photoinitiator is one of 1-hydroxycyclohexylphenylketone, 2,4-dihydroxybenzophenone, α-diethoxy acetophenone, and benzophenone.

4. The UV double-sided adhesive according to claim 1, wherein the surfactant is disproportionated rosin potassium soap or maleic rosin.

5. The UV double-sided adhesive according to claim 1, wherein preparation steps of the fluorine modified core-shell polyacrylic acid modified resin are as follows:
S1: adding a hybrid emulsifier and deionized water into a round bottom flask, stirring at room temperature, dropping 20 g of core material into the flask for 5 min, stirring vigorously for 1 h, and obtaining a pre-polymerized core material lotion; preparing a pre-polymerized modified shell material lotion with the same experimental procedure;
S2: adding SR-10 emulsifier, AEO-9 emulsifier, NaHCO₃, ammonium persulfate into a mixture composed of the pre-polymerized core material lotion and deionized water; stirring at 80° C. and 220 r/min to synthesis a seed lotion, and keeping at 85° C. for 30 min; dropping the pre-polymerized modified shell material lotion to the seed lotion until to be blue, and staying at 85° C. for 30 min; dropping the pre-polymerized modified shell material lotion and an aqueous solution of initiator with the same experimental procedure, cooling to room temperature to obtain the fluorine modified core-shell polyacrylic acid modified resin.

6. The preparation steps of the fluorine modified core-shell polyacrylic acid modified resin according to claim 5, wherein the core material is one of methyl methacrylate, butyl acrylate, or acrylic acid; the modified shell material is dodecafluoroheptyl methacrylate.

7. The preparation steps of the fluorine modified core-shell polyacrylic acid modified resin according to claim 5, wherein in S1, a mass ratio of SR-10 emulsifier to AEO-9 emulsifier in the hybrid emulsifier is 2:1,
a mass ratio of hybrid emulsifier, deionized water, and core material is 2:3:1;
in S2, a mass ratio of SR-10 emulsifier, AEO-9 emulsifier, NaHCO₃, ammonium persulfate, pre-polymerized modified shell material lotion and deionized water is 0.25:0.13:0.2:0.15:6.5:44.

8. The UV double-sided adhesive according to claim 1, wherein preparation steps of the UV curing adhesive are as follows:
S1: adding 5-6 parts by weight of fluorine modified core-shell polyacrylic acid modified resin, 25-30 parts by weight of polyurethane acrylic resin, 25-30 parts by weight of tackifying acrylic resin, 8.3-10 parts by weight of isobornyl acrylate, and 16.7-20 parts by weight of ethylhexyl acrylate to a reactor to obtain a pre-polymerized resin blend compound;
S2: adding 1-3 parts by weight of surfactant dropwise to the pre-polymerized resin blend compound at 0-5° C., homogenizing for 30 minutes, heating up to 50-60° C. and refluxing for 6 hours, adding 0.83-1 parts by weight of antioxidant and 1.67-2 parts by weight of coupling agent, reacting for 3-4 hours at 90-120° C., cooling to 50-60° C., and obtaining a crude adhesive;
S3: adding 2.5-3 parts by weight of photoinitiator and water to the crude adhesive for compounding, homogenizing for 30 minutes, and preparing the UV curing adhesive.

9. The UV double-sided adhesive according to claim 1, wherein a production environment configuration: a production workshop needs to be equipped with a yellow light to shield natural light and UV light sources.

10. A preparation method of the UV double-sided adhesive according to claim 1, comprising the following steps:
S1: pouring evenly mixed glue into a UV curing coating machine, turning on UV curing lamp, adjusting light energy to 500-600 mJ, setting a feeding speed to 3.1 m/min, evenly applying the glue onto the first PET release film by a machine, forming a glue layer after the glue is solidified for a first time, and covering the second PET release film on the other side of the glue layer;
S2: material winding, rolling into a film with a width of 1000 mm and a length of 200 m by a winding machine;
S3: cutting the film into small rolls and feeding them onto a die-cutting machine for die-cutting into small pieces.
